(19) United States
(12) Patent Application Publication
(10) Pub. No.: US 2026/0085273 A1
(43) Pub. Date: Mar. 26, 2026

(54) METHOD AND SYSTEM OF COMMUNICATION BETWEEN A COMMUNICATION DEVICE ASSOCIATED WITH A BIOREACTOR CONTROLLER FOR CONTROLLING A BIOREACTOR AND ONE OR MORE OTHER COMMUNICATION DEVICES

(71) Applicant: Applikon Biotechnology B.V., Delft (NL)

(72) Inventors: Wilbert Timotheüs WALVOORT, Delft (NL); Leo VEENSTRA, Delft (NL)

(21) Appl. No.: 19/397,572

(22) Filed: Nov. 21, 2025

Related U.S. Application Data
(63) Continuation of application No. PCT/NL2024/050263, filed on May 22, 2024.

(30) Foreign Application Priority Data
May 22, 2023 (NL) 2034877

(51) Int. Cl. C12M 1/36; H04L 67/12

(52) U.S. Cl. CPC C12M 41/48; H04L 67/12

(57) ABSTRACT

The disclosure concerns a method of communication between a communication device comprised by a bioreactor controller for controlling a bioreactor and one or more other communication devices, comprising the step of:
communicating a message between the communication device comprised by the bioreactor controller and the one or more other communication devices over a communications network communicatively connecting the communication device to the one or more other communication devices,
wherein the message comprises a human-readable message between an operator operating the communication device comprised by the bioreactor controller and one or more other operators operating the one or more other communication devices,
wherein the message communicated between the operator and the one or more other operators is effectuated by the operator or the one or more other operators.

### CROSS-REFERENCE TO RELATED APPLICATIONS

This application is a continuation under 35 U.S.C. § 120 of International Application No. PCT/NL2024/050263, filed May 22, 2024, which is claims priority to Netherlands Application No. NL 2034877, filed May 22, 2023, under 35 U.S.C. § 119(a). Each of the above-referenced patent applications is incorporated by reference in its entirety.

### FIELD

The present disclosure relates to a method of communication between (a) a communication device comprised by a bioreactor controller for controlling a bioreactor and (b) one or more other communication devices, as well as a communication device, a bioreactor controller, a bioreactor system, a communication system, a computer system and a non-transitory computer-readable medium for use with such a method.

### BACKGROUND

A bioreactor generally relates to a manufactured or engineered device or system that supports a biologically active environment. A bioreactor may also be broadly regarded as an artificial (so non-natural) environment for cell growth. Such a bioreactor usually comprises a vessel in which a biological process is carried out which involves organisms or biochemically active substances derived from such organisms. This process can either be aerobic or anaerobic.

A bioreactor may also refer to a device or system designed to grow cells or tissues in the context of cell culture. Such devices are being developed for use in tissue engineering or biochemical engineering.

Based on their mode of operation, a bioreactor may be classified as batch, fed batch or continuous. Organisms growing in bioreactors may be submerged in liquid medium or may be attached to the surface of a solid medium. Submerged cultures may be suspended or immobilized.

In a continuously operated process the organisms will remain in the reactor and byproducts will be removed from the reactor with the effluent. A typical bioreactor comprises the following parts: an agitator or impeller—used for the mixing of the contents of the reactor which keeps the “cells” in the perfect homogenous condition for better transport of nutrients and oxygen to the cells, a baffle—used to break the vortex formation in the vessel, a sparger—to supply adequate gasses (such as oxygen or CO2₎ to the growing cells, and a jacket—the jacket provides the annular area for circulation of certain temperature of water which keeps the temperature of the bioreactor at a desired value, although the use of Peltier heating or heating blankets is also conceivable. Bioreactors generally are classified as having a volume of more than 20 liters or a volume of less than 20 liters.

Bioreactor controllers are provided/supplied with one or more bioreactors. Bioreactor controllers contain electronics and software to read from the sensors in the bioreactor. Such bioreactor controllers also contain software to interpret the sensor signals and, based on human preset control parameters, control the bioreactor actuators, such as the impeller motor, the liquid addition pumps, gas flow additions, et cetera. Bioreactor controllers provide an interface, typically in the form of a touch screen or connection to a PC, for the human operator to monitor the bioprocess in the one or more bioreactors connected to the (one or more) bioreactor controller(s). Bioreactor controllers come in many different shapes and sizes, to match the requirements of the bioreactor. A bioreactor controller may be a single cabinet, or a combination of interconnected modular cabinets. The one or more bioreactors, controlled by the one or more bioreactor controllers, are together referred to as a bioreactor system.

The bioreactor controller may be configured as a single or dual bioreactor control system to match bioprocess requirements.

US 2021/324317 A1 illustrates an example bioreactor usable with the embodiments of the present disclosure, and is herewith incorporated by reference in its entirety.

Monitoring a bioreactor from a location away from the bioreactor, for example to track parameters, such as temperature, dissolved O₂ concentration and dissolved CO₂ concentration, is known in the art. System-generated messages, e.g. alarms, may be sent from the bioreactor system to a user, for instance when an operational parameter, say temperature, exceeds a certain predefined threshold.

Moreover, control system technology is available permitting the linking of bioreactors by e.g. Ethernet for automation purposes, and possibly connecting the bioreactors to a supervision system, for instance for exerting centralized supervisory control.

EP 1598415 A1 discloses a system for cultivating cells in cell culture vessels. The system includes a liquid handling module, an incubator module, a testing module for performing measurement upon cells and/or media and generating output data, a manipulator module, and a workflow management module for controlling the execution of processes within the system. The system may include a means for resolving resource conflicts including a user interface capable of receiving operator input in order to resolve resource conflicts and representing resource conflicts on the display means.

Messaging or sharing content by or between an operator of a bioreactor system to users or operators of other devices, such as laptops, desktops or smartphones, however, is currently impossible. Basically, the operator has to export or download different types of content from the bioreactor system for subsequent communication through communication systems like e-mail, Teams, chat, WhatsApp, et cetera. This takes a relatively large amount of time and effort and may lead to errors. Sometimes such communication is not even possible, when e.g. large size experiment data files are to be sent. Furthermore, some communication is currently not performed adequately or even omitted completely, because of the amount of time and effort involved.

An object of the present disclosure is thus to provide a method for messaging or sharing content between an operator of a bioreactor system and one or more other operators or users, in particular real-time messaging or sharing.

### SUMMARY

Thereto, according to the present disclosure, a method of communication between a communication device comprised by a bioreactor controller for controlling a bioreactor and one or more other communication devices is provided, comprising the step of:
communicating a message between the communication device comprised by the bioreactor controller and the one or more other communication devices over a communications network communicatively connecting the communication device to the one or more other communication devices,
wherein the message comprises a human-readable message between an operator operating the communication device comprised by the bioreactor controller and one or more other operators operating the one or more other communication devices,
wherein the message communicated between the operator and the one or more other operators is effectuated by the operator or the one or more other operators.

The above method allows for direct communication between the operator of the communication device comprised by the bioreactor controller, in particular an operator operating the bioreactor controller, and the operators or users of other communication devices over the communications network, removing the need for the operator to e.g. make an export and for example send the export by e-mail, which saves time and prevents errors. The abovementioned method is particularly suitable for sending human-readable messages or sharing content for operators or users of interconnected bioreactor controllers or bioreactor systems. “Comprised by” means that the communication device is e.g. integrated with, included in/by, or enclosed by the bioreactor controller. The communication device may also be implemented in, or form part of, the hardware of the bioreactor controller, and/or may be installed in, or included in, a housing of the bioreactor controller. The skilled person furthermore knows that controlling may e.g. also comprise the monitoring of the bioreactor.

The one or more other communication devices are preferably comprised by one or more other bioreactor controllers for controlling one or more other bioreactors.

The message communicated between the operator and the one or more other operators may be effectuated by having the operator push or contact one or more buttons or screens comprised by the bioreactor controller and/or by having the one or more other operators push or contact one or more buttons or screens (or, in general, a user-operable control element) comprised by the one or more other bioreactor controllers, allowing for fast and efficient communication between the one or more users or operators of the one or more other communication devices.

The message may further comprise any personal inquiries (‘how was the weekend?’), or any other combination of human-readable characters, including (but not limited to) emoticons.

The message may comprise at least one of a screenshot of the one or more screens comprised by the bioreactor controller and/or the one or more other bioreactor controllers, bioreactor experiment data, one or more bioreactor instrument configurations, one or more dashboard configurations, one or more theme configurations and one or more recipes.

The message may comprise information associated with the operation of the bioreactor and/or the operation of the one or more other bioreactors, such as one or more bioreactor process parameters, for example temperature, acidity (pH), dissolved O₂ concentration, dissolved CO₂ concentration, et cetera.

The information associated with the operation of the bioreactor and/or the operation of the one or more other bioreactors preferably is real-time information, such as one or more real-time bioreactor process parameters. Thus, real-time communication between the operator and other operators or users is facilitated.

The one or more other communication devices may also be comprised by one or more computing devices, such as a smartphone, laptop or desktop, e.g. being integrated therewith. It is conceivable that the operator is using a computing device, e.g. her/his phone or tablet, as a direct interface to control the bioreactor controller and will then send/receive a message with her/his phone or tablet, through the communications network, e.g. a network of connected bioreactor systems, to another operator in the communications network, e.g. the bioreactor network. Such a communication device directly interfacing with the bioreactor controller is also to be regarded as “comprised by” the bioreactor controller.

As mentioned before, the bioreactor controller comprises the communication device, i.e. the communication device is part of or forms a portion or section of the bioreactor controller, and/or the one or more other bioreactor controllers may analogously comprise the one or more other communication devices. The communication device may be implemented in, or form part of, the hardware of the bioreactor controller.

The communication device comprised by the bioreactor controller and/or the one or more other communication devices comprised by the one or more other bioreactor controllers, are communicatively connected to a server via the communications network, wherein the server is arranged for controlling the communicating of the message between the communication device comprised by the bioreactor controller and the one or more other communication devices over the communications network. The server may comprise software functionality to facilitate and help the operators with creating the messages, such as spelling correction, word/sentence suggestions, et cetera. Such functionality may comprise Artificial Intelligence (AI).

The server may also store and or process, and or format and/or analyze data from the one or more bioreactors connected to the one or more bioreactor controllers connected to the server, and the messages may refer to (segments of) such data/analysis on the server.

The server may also hold the history of messages sent, which might also redundantly be stored on the one or more bioreactor controllers.

The server may be able to connect to external servers, such as those of other companies or individuals, such as Getinge or Microsoft or LinkedIn, for a.o. account synchronization. The server may contain one or more databases, such as one for the experiment data and/or message data and/or account data. The server may employ several applications for the different functionalities. The server may also manage and control the licenses for the bioreactor controller system and/or server functionalities, possibly through a connection to external servers (such as Getinge). The server may contain redundant hardware, such as multiple devices for storage and/or processors and/or power supply. Two or more server instances may be set up in the network for additional redundancy. The one or more servers may have their own interfaces and/or may be reachable through the other interfaces in the network. The server (functionality) may be provided on external hardware through an internet connection (i.e. a Cloud). The server may provide connection to a market place, such as one for bioreactor and/or bioreactor controller and/or bioreactor system and/or bioreactor network functionalities.

The communication device comprised by the bioreactor controller and the one or more other communication devices comprised by the one or more bioreactor controllers may be part of a network of connected bioreactor controllers or bioreactor systems, wherein the server is configured for synced or centralized, personalized user login throughout the network of connected bioreactor controllers or bioreactor systems. It should be noted that messaging often requires centralized and personalized accounts. Common general knowledge of bioreactor systems does not include such a network of bioreactor systems having centralized and personalized accounts.

Another aspect of the disclosure concerns a communication device, configured for use with the aforementioned method, wherein (a) the communication device is comprised by the bioreactor controller for controlling the bioreactor and/or wherein the communication device is configured for use as one of the (b) one or more other communication devices.

Another aspect of the disclosure concerns a bioreactor controller for controlling a bioreactor, comprising an aforementioned communication device.

Another aspect of the disclosure concerns a bioreactor system, comprising a bioreactor and an aforementioned bioreactor controller.

Another aspect of the disclosure concerns a communication system, comprising an aforementioned bioreactor controller or an aforementioned bioreactor system, the one or more other communication devices, and a communications network communicatively connecting the communication device to the one or more other communication devices.

As mentioned before, the one or more other communication devices may be comprised by one or more other bioreactor controllers for controlling one or more other bioreactors.

The aforementioned communication system may comprise the one or more other bioreactor systems with the one or more other bioreactors and the one or more other bioreactor controllers for controlling the one or more other bioreactors.

Another aspect of the disclosure concerns a computer system, comprising:
one or more computer system processors; and
memory storing instructions that are executable by the one or more computer system processors to perform the aforementioned method.

Another aspect of the disclosure concerns a computer software program, comprising instructions that are executable by one or more computer system processors for performing the aforementioned method.

Another aspect of the disclosure concerns a non-transitory computer-readable medium comprising instructions that are executable by one or more computer system processors for performing the aforementioned method.

The aforementioned computer system, computer software program according and/or computer-readable medium may be comprised by the aforementioned communication device and/or the aforementioned bioreactor controller.

### BRIEF DESCRIPTION OF THE DRAWINGS

The disclosure will be explained in more detail below, with reference to illustrative embodiments shown in the drawings. Therein:

FIG. 1 shows an example embodiment of a network of connected bioreactor systems for performing an example embodiment of the method according to the present disclosure, with a server configured for synced or centralized, personalized user login throughout the network of connected bioreactor systems;

FIG. 2 shows an example embodiment of a bioreactor system for performing an example embodiment of the method according to the present disclosure;

FIG. 3 shows an example embodiment of a (screenshot of a) screen associated with an example embodiment of a bioreactor controller according to the present disclosure,

FIG. 4 shows an example embodiment of a screen associated with an example embodiment of a bioreactor controller according to the present disclosure, showing messages sent and received between the one operator and one or more other operators or users, and

FIG. 5 shows an example embodiment of one or more computer systems for performing an example embodiment of the method according to the present disclosure.

### DETAILED DESCRIPTION

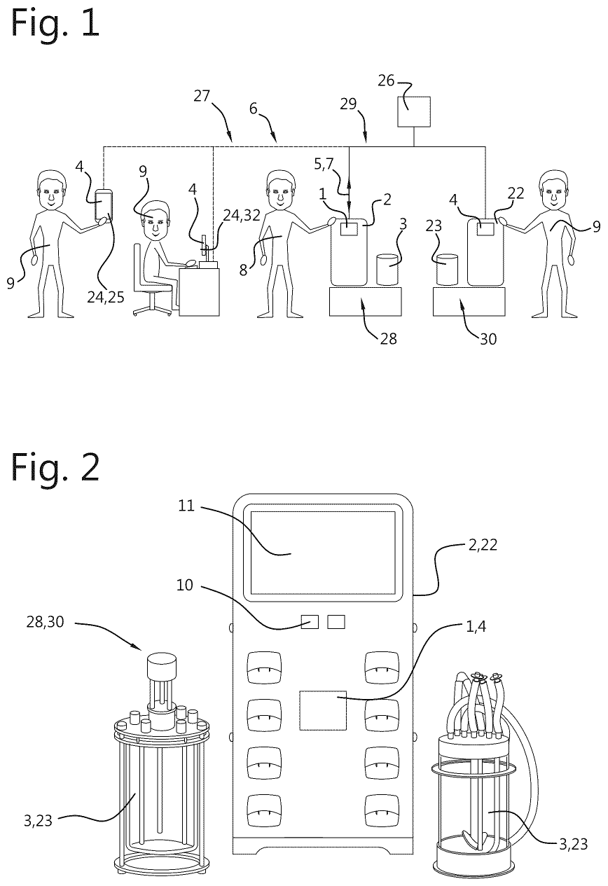

FIGS. 1-5 will be discussed in conjunction. As mentioned in the foregoing, FIG. 1 shows an example embodiment of a network 27 of connected bioreactor systems 28, 30 for performing an example embodiment of the method according to the present disclosure, with a server 26 configured for synced or centralized, personalized user login throughout the network 27 of connected bioreactor systems 28, 30. Many other embodiments are conceivable though for performing the method according to the present disclosure. Each bioreactor system 28, 30, comprises a bioreactor 3, 23 and a bioreactor controller 2, 22. The network 27 may for instance comprise two, three, four, or more connected bioreactor systems 28, 30 and a corresponding amount of bioreactor controllers 2, 22. The bioreactors 3, 23 may have any conceivable form (e.g. single-use or multi-use) and are preferably communicatively connected to the bioreactor controllers 2, 22 for exchanging data, control commands, et cetera. The bioreactor systems 28, 30 may for instance be positioned on a bench or the like, as shown in FIG. 1.

According to the present disclosure, a method of communication between a communication device 1 comprised by a bioreactor controller 2 for controlling a bioreactor 3 and one or more other communication devices 4 is provided. The method comprises the step of communicating a message 5 between the communication device 1 comprised by the bioreactor controller 2 and the one or more other communication devices 4 over a communications network 6 communicatively connecting the communication device 1 to the one or more other communication devices 4. The communications network 6 may comprise a LAN or WAN (e.g. cloud) network, and may be implemented by means of Ethernet cabling, Wi-Fi, 5G, et cetera. The message 5 comprises a human-readable message 7, for instance comprising text or graphics, between an operator 8 operating the communication device 1 comprised by the bioreactor controller 2 and one or more other operators or users 9 operating the one or more other communication devices 4. The message 5 to be communicated between the operator 8 and the one or more other operators 9, such as communication comprising sending or receiving, is (configured to be) effectuated by the operator 8 or the one or more other operators 9. Some of the one or more other communication devices 4 as shown in FIG. 1 are comprised by d with one or more other bioreactor controllers 22 for controlling one or more other bioreactors 23. The one or more other communication devices 4, as shown in the left part of FIG. 1, may be comprised by one or more computing devices 24, such as a smartphone 25, laptop or desktop 32, and users or operators 9 thereof.

As shown in the example embodiment of FIG. 1, the bioreactor controller 2 comprises the communication device 1 and one or more other bioreactor controllers 22 comprise one or more other communication devices 4.

As shown in the example embodiment of FIG. 1, the communication device 1 comprised by the bioreactor controller 2 and the one or more other communication devices 4 comprised by the one or more other bioreactor controllers 22, are communicatively connected to a server 26 via the communications network 6. The server 26 is arranged for controlling the communicating of the message 5 between the communication device 1 comprised by the bioreactor controller 2 and the one or more other communication devices 4 over the communications network 6.

The communication device 1 comprised by the bioreactor controller 2 and the one or more other communication devices 4 comprised by the one or more bioreactor controllers 22 as shown in FIG. 1 are part of a network 27 of connected bioreactor controllers 2, 22 or bioreactor systems 28, 30. The server 26 therein is configured for synced or centralized, personalized user login throughout the network 27 of connected bioreactor controllers 2, 22 or bioreactor systems 28, 30.

FIG. 1 thus also generally shows a communication system 29 according to the present disclosure, comprising a communication device 1, a bioreactor controller 2 or a bioreactor system 28, the one or more other communication devices 4, and a communications network 6 communicatively connecting the communication device 1 to the one or more other communication devices 4. As mentioned before, the one or more other communication devices 4 may be comprised by the one or more other bioreactor controllers 22 for controlling one or more other bioreactors 23. The communication system 29 may comprise the one or more other bioreactor systems 30 with the one or more other bioreactors 23 and the one or more other bioreactor controllers 22 for controlling the one or more other bioreactors 22.

FIG. 2 shows an example embodiment of a bioreactor system 28, 30 for performing an example embodiment of the method according to the present disclosure. Preferably, the operator 8 or the one or more other operators 9 effectuate the message 5 to be communicated between the operator 8 and the one or more other operators 9 by pushing or contacting one or more buttons 10 or one or more screens 11 comprised by the bioreactor controller 2, 22. In the embodiment depicted in FIG. 2, the communication device 1, 4 is comprised by, e.g. integrated with the bioreactor controller 2, 22, such as in the hardware thereof. As shown in FIG. 2, the bioreactor system 28, 30 may comprise a multi-use bioreactor 3, 23 (as shown to the left of the bioreactor controller 2, 22) or a single-use bioreactor 3, 23 (as shown to the right of the bioreactor controller 2, 22). The screen 11 may e.g. comprise a touchscreen, for instance with a message button, share button and/or chat button, so that an operator/user 8, 9 can send a message and/or share content. Physical buttons 10 could also be used, as shown in FIG. 2. An operator/user 8, 9 may also connect a tablet, smartphone or the like to the bioreactor controller 2, 22, for instance through a suitable data communication cable, such as a USB cable, or through a wireless communication method, such as NFC, Bluetooth or Wi-Fi, as interface to control the bioreactor controller 2, 22 and will then may send/receive a message 5, 7 with her/his phone or tablet. Such a communication device 1, 4 directly interfacing with the bioreactor controller 2, 22 is also to be regarded as “comprised by” the bioreactor controller 2, 22.

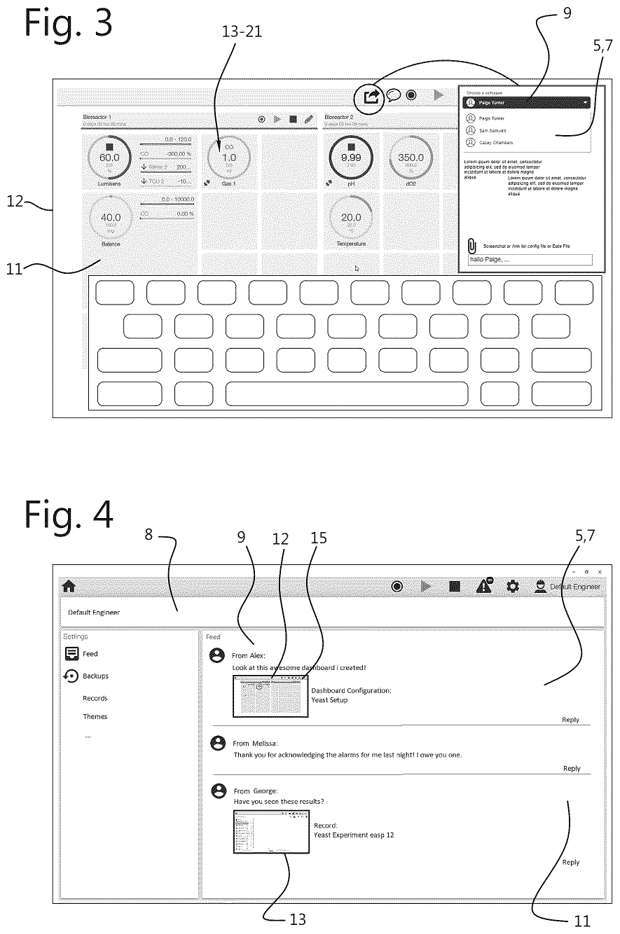

FIG. 3 shows an example embodiment of a (screenshot 12 of a) screen 11 comprised by an example embodiment of a bioreactor controller 2, 22 according to the present disclosure, such as the bioreactor controller 2, 22 shown in FIG. 2. The screen 11 shows a human-readable message 5, 7 to be sent to another operator or user 9, indicated by name. The message 5, 7 may for instance comprise an attachment, as indicated by the paperclip in the lower portion of the message box/menu. The message 5, 7 may e.g. comprise one of a screenshot 12 of the one or more screens 11 comprised by the bioreactor controller 2, 22, bioreactor experiment data 13, one or more bioreactor instrument configurations 14, one or more dashboard configurations 15, one or more theme configurations 16 and one or more recipes 17. The message 5 may also comprise information 18 associated with the operation of the bioreactor 3, 23, such as one or more bioreactor process parameters 19. The information 18 associated with the operation of the bioreactor 3, 23 may be real-time information 20, such as one or more real-time bioreactor process parameters 21. The screen 11 may e.g. comprise a virtual keyboard or the like so the operator 8 can easily enter a human-readable message 7, such as an inquiry or question.

FIG. 4 shows an example embodiment of a screen 11 comprised by an example embodiment of a bioreactor controller 2, 22 according to the present disclosure, showing a message box with messages 5, 7 sent and received between the one operator 8, called “Default Engineer”, and one or more other operators or users 9. The upper message 5, 7 sent to the operator 8 by another operator 9 called Alex for instance comprises a screenshot 12 of a dashboard configuration 15, as well as human-readable text. The middle message 5, 7 sent by another operator 9 called Melissa merely comprises human-readable text. The lower message 5, 7 sent by another operator 9 called George comprises bioreactor experiment data 13, for instance in the form of a graph or table, as well as human-readable text. A “reply” button is shown allowing the operator 8 to instantly reply to incoming messages 5, 7.

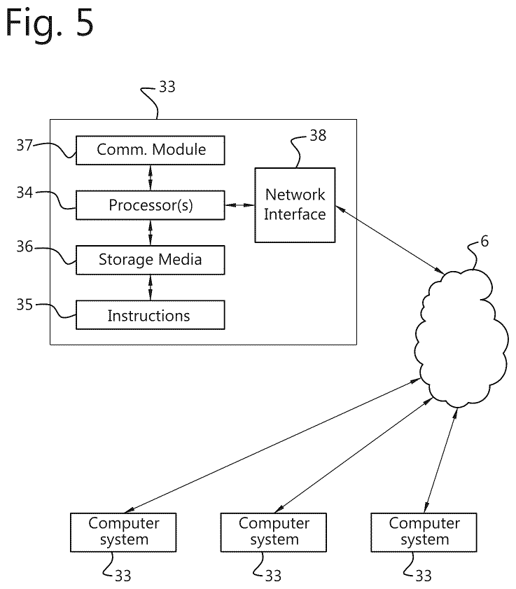

FIG. 5 illustrates an example of a computer system 33, which may be an individual computer system 33 or an arrangement of distributed computer systems 33, for performing the method according to the present disclosure. The computer system 33 may be comprised by a communication device 1, 4 or a bioreactor controller 2, 22. Another aspect of the disclosure relates to a non-transitory computer-readable medium 36 comprising instructions 35 that are executable by one or more computer system processors 34 for performing the method according to the disclosure. Another aspect of the disclosure relates to a computer software program, comprising instructions 35 that are executable by one or more computer system processors 34 for performing the method according to the present disclosure.

The computer system 33 includes one or more communications module(s) 37 configured to perform various tasks according to some embodiments, such as one or more methods disclosed herein. To perform these various tasks, the communications module 37 executes independently, or in coordination with, one or more processors 34, which is (or are) connected to one or more storage media 36. The processor(s) 34 is (or are) also connected to a network interface 38 to allow the computer system 33 to communicate over a data/communications network 6 with one or more additional computer systems 33, e.g. comprised by the other communication devices 4 or other bioreactor controllers 22 (note that computer systems 33 may or may not share the same architecture, and may be located in different physical locations, e.g., some computer systems 33 may be located in a lab/processing facility, while in communication with one or more other computer systems 33 that are located in one or more data centers, and/or located in varying countries on different continents).

A processor 34 can include a microprocessor, microcontroller, processor module or subsystem, programmable integrated circuit, programmable gate array, or another control or computing device.

The storage media 36 can be implemented as one or more computer-readable or machine-readable storage media 36. Note that while in the example embodiment of FIG. 5 storage media 36 is depicted as within computer system 33, in some embodiments, storage media 36 may be distributed within and/or across multiple internal and/or external enclosures of computer system 33 and/or additional computer systems 33. Storage media 36 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital versatile disks (DVDs), BLU-RAY® disks, or other types of optical storage, or other types of storage devices. Note that the instructions 35 discussed above can be provided on one computer-readable or machine-readable storage medium 36, or in another embodiment, can be provided on multiple computer-readable or machine readable storage media distributed in a large system having possibly plural nodes. Such computer-readable or machine-readable storage medium 36 or media 36 is (are) considered to be part of an article (or article of manufacture).

An article or article of manufacture can refer to any manufactured single component or multiple components. The storage medium 36 or media 36 can be located either in the machine running the machine-readable instructions, or located at a remote site from which machine-readable instructions can be downloaded over a network for execution.

It should be appreciated that computer system 33 may have more or fewer components than shown, may combine additional components not depicted in the example embodiment of FIG. 5, and/or computer system 30 may have a different configuration or arrangement of the components depicted in FIG. 5. The various components shown in FIG. 5 may be implemented in hardware, software, or a combination of both hardware and software, including one or more signal processing and/or application specific integrated circuits.

The various components shown in FIG. 5, such as a computer system 33, may e.g. be implemented in the hardware (and/or software) of the communication device 1, 4 or the bioreactor controller 2, 22.

Further, the processing methods described herein may be implemented by running one or more functional modules in information processing apparatus such as general purpose processors or application specific chips, such as ASICs, FPGAs, PLDs, or other appropriate devices. These modules, combinations of these modules, and/or their combination with general hardware are all included within the scope of protection of the disclosure, as defined by the appended claims.

Although the disclosure has been described above with reference to example embodiments, variants within the scope of the present disclosure will readily occur to those skilled in the art after reading the above description. Such variants are within the scope of the independent claims and the dependent claims. In addition, it is to be understood that express rights are requested for variants as described in the dependent claims. It should also be noted that the example embodiments shown in the Figures, or features thereof, may be combined to yield embodiments not explicitly shown in the Figures.

### list of reference numerals

1. Communication device
2. Bioreactor controller
3. Bioreactor
4. Other communication device
5. Message
6. Communications network
7. Human-readable message
8. Operator
9. Other operator
10. Button comprised by the bioreactor controller
11. Screen comprised by the bioreactor controller
12. Screenshot
13. Bioreactor experiment data
14. Bioreactor instrument configuration
15. Dashboard configuration
16. Theme configuration
17. Recipe
18. Information associated with the operation of the bioreactor
19. Bioreactor process parameters
20. Real-time information associated with the operation of the bioreactor
21. Real-time bioreactor process parameters
22. Other bioreactor controller
23. Other bioreactor
24. Computing device
25. Smartphone
26. Server
27. Network of connected bioreactor systems
28. Bioreactor system
29. Communication system
30. Other bioreactor system
31. -
32. Desktop
33. Computer system
34. Computer system processor
35. Instructions
36. Computer-readable storage medium
37. Communications module
38. Network interface

1. Method of communication between (a) a communication device (1) comprised by a bioreactor controller (2) for controlling a bioreactor (3) and (b) one or more other communication devices (4), comprising the step of:
communicating a message (5) between the communication device comprised by the bioreactor controller and the one or more other communication devices over a communications network (6) communicatively connecting the communication device to the one or more other communication devices,
wherein the message (5) comprises a human-readable message (7) between an operator (8) operating the communication device (1) comprised by the bioreactor controller (2) and one or more other operators (9) operating the one or more other communication devices (4),
wherein the message (5) communicated between the operator and the one or more other operators (9) is effectuated by the operator (8) or the one or more other operators.

2. Method according to claim 1, wherein one or more other communication devices (4) are comprised by one or more other bioreactor controllers (22) for controlling one or more other bioreactors (23).

3. Method according to claim 1 or 2, wherein the message (5) communicated between the operator (8) and the one or more other operators (9) is effectuated by having the operator (8) push or contact one or more buttons (10) or screens (11) comprised by the bioreactor controller (2) and/or, when dependent on claim 2, by having the one or more other operators (9) push or contact one or more buttons (10) or screens (11) comprised by the one or more other bioreactor controllers (22).

4. Method according to any one of the preceding claims, wherein the message (5) comprises at least one of a screenshot (12) of the one or more screens (11) comprised by the bioreactor controller (2) and/or, when dependent on claim 3, the one or more other bioreactor controllers (22), bioreactor experiment data (13), one or more bioreactor instrument configurations (14), one or more dashboard configurations (15), one or more theme configurations (16) and one or more recipes (17).

5. Method according to any one of the preceding claims, wherein the message (5) comprises information (18) associated with the operation of the bioreactor (3), and/or, when dependent on claim 2, the operation of the one or more other bioreactors (23), such as one or more bioreactor process parameters (19).

6. Method according to claim 5, wherein the information (18) associated with the operation of the bioreactor (3) and/or the operation of the one or more other bioreactors (23) is real-time information (20), such as one or more real-time bioreactor process parameters (21).

7. Method according to any one of the preceding claims, wherein one or more other communication devices (4) are comprised by one or more computing devices (24), such as a smartphone (25), laptop or desktop (32).

8. Method according to any one of the preceding claims, wherein the communication device (1) comprised by the bioreactor controller (2) and/or, when dependent on claim 2, the one or more other communication devices (4) comprised by the one or more other bioreactor controllers (22), are communicatively connected to a server (26) via the communications network (6), wherein the server is arranged for controlling the communicating of the message (5) between the communication device (1) comprised by the bioreactor controller (2) and the one or more other communication devices (4) over the communications network (6).

9. Method according to claim 2 and 8, wherein the communication device (1) comprised by the bioreactor controller (2) and the one or more other communication devices (4) comprised by the one or more bioreactor controllers (22) are part of a network (27) of connected bioreactor controllers (2, 22) or bioreactor systems (28, 30), wherein the server (26) is configured for synced or centralized, personalized user login throughout the network of connected bioreactor controllers or bioreactor systems.

10. Communication device (1, 4), configured for use with the method according to any one of the preceding claims, wherein (a) the communication device (1) is comprised by the bioreactor controller (2) for controlling the bioreactor (3) and/or wherein the communication device (4) is configured for use as one of the (b) one or more other communication devices (4).

11. Bioreactor controller (2, 22) for controlling a bioreactor (3, 23), comprising a communication device (1, 4) according to claim 10.

12. Bioreactor system (28, 30), comprising a bioreactor (3, 23) and a bioreactor controller (2, 22) according to claim 11.

13. Communication system (29), comprising a bioreactor controller (2) according to claim 11 or a bioreactor system (28) according to claim 12, the one or more other communication devices (4), and a communications network (6) communicatively connecting the communication device (1) to the one or more other communication devices (4).

14. Communication system (29) according to claim 13, wherein the one or more other communication devices (4) are comprised by one or more other bioreactor controllers (22) for controlling one or more other bioreactors (23).

15. Communication system (29) according to claim 14, comprising the one or more other bioreactor systems (30) with the one or more other bioreactors (23) and the one or more other bioreactor controllers (22) for controlling the one or more other bioreactors.

16. Computer system (33), comprising:
one or more computer system processors (34); and
memory storing instructions (35) that are executable by the one or more computer system processors to perform the method according to any one of the claims 1-9.

17. Computer software program, comprising instructions (35) that are executable by one or more computer system processors (34) for performing the method according to any one of the claims 1-9.

18. Non-transitory computer-readable medium (36) comprising instructions (35) that are executable by one or more computer system processors (34) for performing the method according to any one of the claims 1-9.

19. Computer system (33) according to claim 16, computer software program according to claim 17 and/or computer-readable medium according to claim 18, comprised by the communication device (1, 4) according to claim 10 and/or the bioreactor controller (2, 22) according to claim 11.
